(19) United States
(12) Patent Application Publication
(10) Pub. No.: US 2017/0303940 A1
(43) Pub. Date: Oct. 26, 2017

(54) PERCUTANEOUS ACCESS DEVICE WITH ADJUSTABLE DEPTH STOP

(71) Applicant: BOSTON SCIENTIFIC LIMITED, Hamilton (BM)

(72) Inventors: ERIK E. SPERRY, Newburyport, MA (US); JERRY T. LONG, JR., Jamaica Plain, MA (US); RONALD CIULLA, Westford, MA (US); TIMOTHY P. HARRAH, Cambridge, MA (US); AARON K. KIRKEMO, Gladstone, NJ (US); KENNETH P. REEVER, Hopedale, MA (US); BRANDON W. CRAFT, Phoenix, MD (US); ELIZABETH A. STOKLEY, Boston, MD (US); SEBASTIAN KOERNER, Berlin (DE); CHAD SCHNEIDER, Owings Mills, MD (US); ANANT SUBRAMANIAM, Owings Mills, MD (US); ASHISH JAIN, Lucknow (IN); DIPANKA MEDHI, Guwahati (IN); SUMIT MALIK, Gurgaon (IN); SUBODH MOREY, Ponda (IN); BITEN K. KATHRANI, Gurgaon (IN); Salman Kapadia, Seoni (IN)

(21) Appl. No.: 15/496,850

(22) Filed: Apr. 25, 2017

Related U.S. Application Data
(60) Provisional application No. 62/372,561, filed on Aug. 9, 2016.
(60) Provisional application No. 62/327,747, filed on Apr. 26, 2016.

(51) Int. Cl. A61B 17/22; A61B 17/34; A61M 25/09; A61M 25/06; A61M 25/00

(52) U.S. Cl. CPC A61B 17/22; A61M 25/09; A61M 25/06; A61B 17/34; A61M 2025/0008; A61M 2025/09166

(57) ABSTRACT

A percutaneous nephrolithotomy (PCNL) needle may include a cannula including a shaft and a cannula hub coupled to a proximal end of the cannula shaft. A depth guide may be disposed on an outer surface of the cannula shaft. A stylet may be disposable within the cannula lumen and may include a tapered point at a distal end of the stylet and a stylet hub coupled to a proximal end of the stylet that is configured to be releasably securable to the cannula hub. An adjustable depth stop may be releasably securable to the cannula shaft at a desired position relative to the depth guide, the adjustable depth guide capable of being manipulated between an adjustment configuration in which the adjustable depth guide is moveable relative to the cannula shaft and a secured configuration in which the adjustable depth guide is secured relative to the cannula shaft.

### CROSS-REFERENCE TO RELATED APPLICATIONS

This application claims the benefit of priority under 35 U.S.C. §119 to U.S. Provisional Application No. 62/327,747, filed Apr. 26, 2016 and U.S. Provisional Application No. 62/372,561, filed Aug. 9, 2016, the entirety of which are incorporated herein by reference.

### TECHNICAL FIELD

The present disclosure pertains generally to medical devices. More particularly, the present disclosure pertains to percutaneous access devices for accessing a target site in a human body.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example medical device is a percutaneous nephrolithotomy (PCNL) needle including a cannula including a shaft defining a cannula lumen extending through the shaft and a cannula hub coupled to a proximal end of the cannula. A depth guide may be disposed on an outer surface of the cannula shaft and may be configured to provide an indication of insertion depth. A stylet may be disposable within the cannula lumen and may include a tapered point at a distal end of the stylet and a stylet hub coupled to a proximal end of the stylet that is configured to be releasably securable to the cannula hub. An adjustable depth stop may be releasably securable to the cannula shaft at a desired position relative to the depth guide, the adjustable depth guide capable of being manipulated between an adjustment configuration in which the adjustable depth guide is moveable relative to the cannula shaft and a secured configuration in which the adjustable depth guide is secured relative to the cannula shaft.

Alternatively or additionally to any of the embodiments above, the PCNL needle further includes a first alignment marker disposed on the cannula hub and a second alignment marker disposed on the stylet hub, the first alignment marker and the second alignment marker positioned to provide a visual indication that the stylet hub is aligned with the cannula hub.

Alternatively or additionally to any of the embodiments above, the PCNL needle further includes a tapered guidewire lumen extending through the cannula hub.

Alternatively or additionally to any of the embodiments above, the stylet has a length sufficient to permit the tapered point of the stylet to extend distally from a distal end of the cannula when the stylet hub is secured to the cannula hub.

Alternatively or additionally to any of the embodiments above, the stylet hub is configured to threadedly engage with the cannula hub.

Alternatively or additionally to any of the embodiments above, the adjustable depth stop includes a radiopaque component.

Alternatively or additionally to any of the embodiments above, the radiopaque component includes a radiopaque ring disposed on the adjustable depth stop.

Alternatively or additionally to any of the embodiments above, the radiopaque component includes a radiopaque material admixed within a polymer forming the adjustable depth stop.

Alternatively or additionally to any of the embodiments above, the radiopaque component includes a radiopaque ink printed onto one or more surfaces of the annular body.

Alternatively or additionally to any of the embodiments above, the adjustable depth stop includes an annular body with an aperture extending through the annular body, the aperture configured to accommodate the cannula therethrough.

Alternatively or additionally to any of the embodiments above, the aperture extending through the annular body is dimensioned to provide a frictional fit with the cannula shaft, such that the annular body may be forceably slid relative to the cannula shaft, but the aperture holds the annular body in place in the absence of force applied to the annular body.

Alternatively or additionally to any of the embodiments above, the PCNL needle further includes a slot formed in the annular body, extending outward from the aperture to a perimeter of the annular body.

Alternatively or additionally to any of the embodiments above, the annular body further includes a cylindrical portion extending concentrically with the cannula shaft, the cylindrical portion including a threaded securement aperture extending through the cylindrical portion orthogonally to the cannula shaft, the adjustable depth stop further comprising a threaded fastener engaged within the threaded securement aperture such that the threaded fastener is capable of being moved into contact with the cannula shaft in order to secure the adjustable depth stop relative to the cannula shaft.

Alternatively or additionally to any of the embodiments above, the adjustable depth stop includes a first leaf having a first aperture sized to accommodate the cannula shaft and a second leaf having a second aperture sized to accommodate the cannula shaft, the first leaf and the second leaf joined via a living hinge.

Alternatively or additionally to any of the embodiments above, the first leaf is biased to a position relative to the second leaf such that the first aperture is at least partially misaligned with the second aperture in order to secure the adjustable depth stop relative to the cannula shaft; and the first leaf and the second leaf are capable of being squeezed together to move the first aperture into alignment with the second aperture such that the adjustable depth stop may be moved relative to the cannula shaft.

Another example medical device includes a percutaneous nephrolithotomy (PCNL) access assembly that includes an access needle having a cannula including a hub and a shaft extending from the hub, a depth guide disposed on an outer surface of the cannula shaft in order to provide an indication of insertion depth and a stylet including a tapered point at a distal end of the stylet and a stylet hub coupled to a proximal end of the stylet, the stylet hub configured to be releasably securable to the cannula hub with the stylet extending through the cannula. An adjustable slider may be releasably securable to the cannula shaft at a desired position relative to the depth guide, the adjustable slider capable of an adjustment configuration in which the adjustable slider is moveable relative to the cannula shaft and a secured configuration in which the adjustable slider is secured relative to the cannula shaft.

Alternatively or additionally to any of the embodiments above, the adjustable slider includes an annular body with an aperture extending through the annular body, the aperture dimensioned to provide a frictional fit with the cannula shaft, such that the annular body may be forceably slid relative to the cannula shaft, but the aperture holds the annular body in place in the absence of force applied to the annular body.

Alternatively or additionally to any of the embodiments above, the annular body further includes a cylindrical portion extending concentrically with the cannula shaft, the cylindrical portion including a threaded securement aperture extending through the cylindrical portion orthogonally to the cannula shaft, the adjustable depth stop further including a threaded fastener engaged within the threaded securement aperture such that the threaded fastener is capable of being moved into contact with the cannula shaft in order to secure the adjustable depth stop relative to the cannula shaft.

Alternatively or additionally to any of the embodiments above, the adjustable slider includes a first leaf having a first aperture sized to accommodate the cannula shaft and a second leaf having a second aperture sized to accommodate the cannula shaft, the first leaf and the second leaf joined via a living hinge; the first leaf is biased to a position relative to the second leaf such that the first aperture is at least partially misaligned with the second aperture in order to secure the adjustable depth stop relative to the cannula shaft; and the first leaf and the second leaf are capable of being squeezed together to move the first aperture into alignment with the second aperture such that the adjustable depth stop may be moved relative to the cannula shaft.

Another example medical device is a percutaneous nephrolithotomy (PCNL) needle including a cannula having a shaft defining a cannula lumen extending through the shaft and a cannula hub coupled to a proximal end of the cannula and a depth guide disposed on an outer surface of the cannula shaft, the depth guide configured to provide an indication of insertion depth. A stylet may be disposable within the cannula lumen and may include a tapered point at a distal end of the stylet and a stylet hub coupled to a proximal end of the stylet, the stylet hub configured to be threadedly engageable with the cannula hub. A first alignment marker may be disposed on the cannula hub and a second alignment marker may be disposed on the stylet hub, the first alignment marker and the second alignment marker positioned to provide a visual indication when the stylet hub is aligned with the cannula hub. An adjustable depth stop may be releasably securable to the cannula shaft at a desired position relative to the depth guide, the adjustable depth guide capable of being manipulated between an adjustment configuration in which the adjustable depth guide is moveable relative to the cannula shaft and a secured configuration in which the adjustable depth guide is secured relative to the cannula shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments of the disclosure in connection with the accompanying drawings, in which:

FIG. 1 is a schematic illustration of a typical kidney, identifying major structures;

FIG. 2 is a side view of a percutaneous nephrolithotomy (PCNL) needle;

FIG. 3 is a perspective view of a stylet forming part of the PCNL needle of FIG. 2;

FIG. 4 is a perspective view of a cannula forming part of the PCNL needle of FIG. 2;

FIG. 5A is a perspective view of a stylet hub forming part of the stylet of FIG. 3;

FIG. 5B is a cross-sectional view of a cannula hub forming part of the cannula of FIG. 4;

FIG. 6 is a perspective view of the PCNL needle of FIG. 2, including an adjustable depth stop;

FIGS. 7 and 8 are views of an adjustable depth stop useable with the PCNL needle of FIG. 2;

FIGS. 9 and 10 are views of an adjustable depth stop useable with the PCNL needle of FIG. 2;

FIGS. 11 to 13 are views of an adjustable depth stop useable with the PCNL needle of FIG. 2;

FIGS. 14 to 17 are views of an adjustable depth stop useable with the PCNL needle of FIG. 2;

FIGS. 18 and 19 are views of an adjustable depth stop useable with the PCNL needle of FIG. 2;

FIGS. 20 and 21 are views of an adjustable depth stop useable with the PCNL needle of FIG. 2;

FIGS. 22 to 24 are views of an adjustable depth stop useable with the PCNL needle of FIG. 2; and

FIGS. 25 and 26 are views of an adjustable depth stop useable with the PCNL needle of FIG. 2.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

There are a variety of medical conditions that may be treated through a percutaneous approach. For example, in some cases kidney stones may be removed via a percutaneous approach. In some cases, kidney stones may be removed using a minimally invasive therapy such as laser therapy or shock wave therapy to break the stones into pieces that are small enough to pass spontaneously out of the body. However, in some instances kidney stone removal may require more invasive therapies. For example, percutaneous nephrolithotomy (PCNL) may be performed to surgically remove a kidney stone. While the devices described herein are described with respect to PCNL and treatment of kidney stones, it will be appreciated that these access devices may be used to treat other forms of disease, including gastrointestinal, airway, urethra, ureter, cardiac, brain, breast, bladder, and peripheral vascular disease, for example. Further, the percutaneous access devices disclosed herein may also be used to access numerous body cavities having both solid and/or hollow organs.

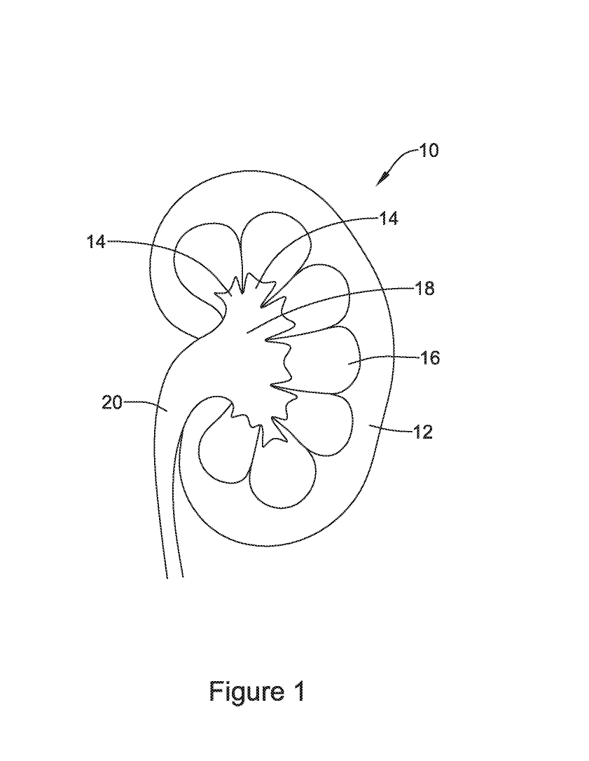

FIG. 1 is a schematic illustration of a typical kidney 10. The kidney 10 includes a renal cortex 12 and several calices 14. In some cases, each calyx 14 includes a medulla 16 and a renal pelvis 18. The calices 14 drain into a ureter 20, which carries urine from the kidney 10 to the patient's urinary bladder (not illustrated). In many cases, a kidney stone, if present, may be located within one of the calyx 14. The number of individual calices 14 within the kidney 10 may vary from individual to individual, although a total of seven are illustrated. Access to the interior of the kidney 10 may be gained via use of a PCNL needle to penetrate the skin and reach the kidney 10. Once the PCNL needle is placed, a guidewire may be extended through the PCNL needle and then additional devices may be extended over the wire. In some cases, an internal component of the PCNL needle may be withdrawn prior guidewire insertion.

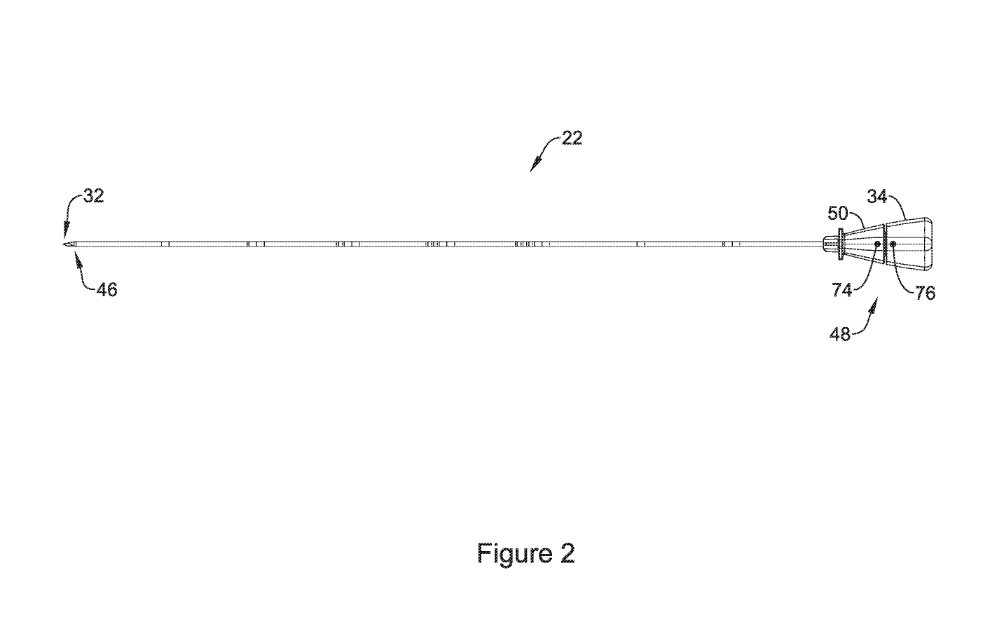

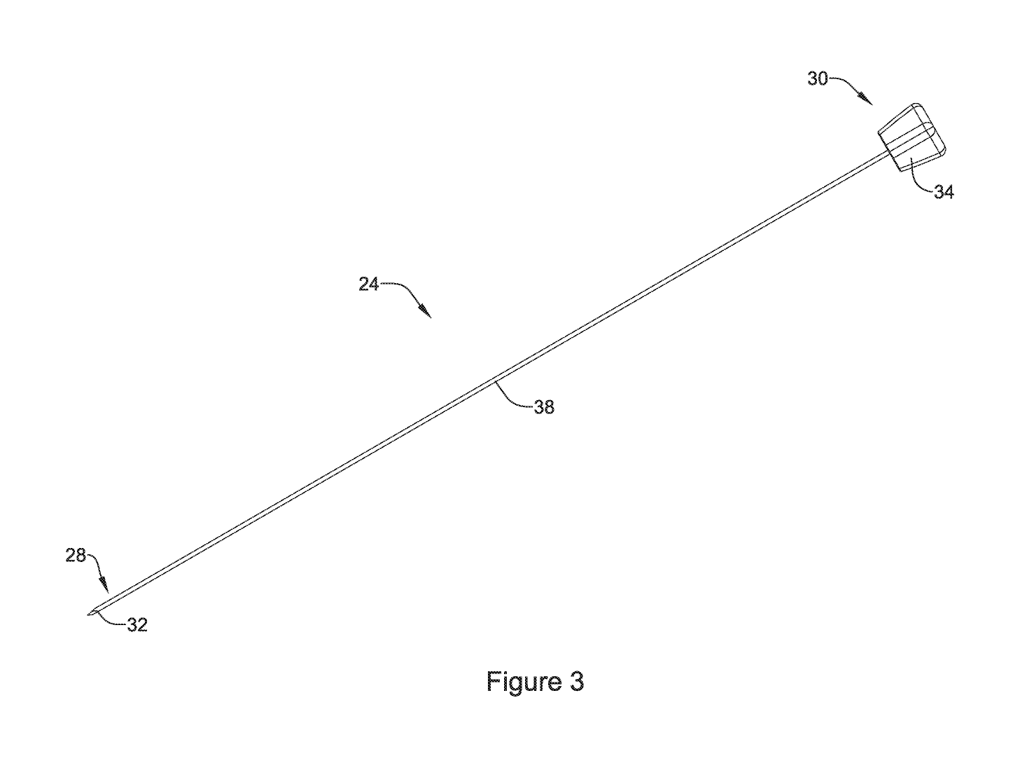

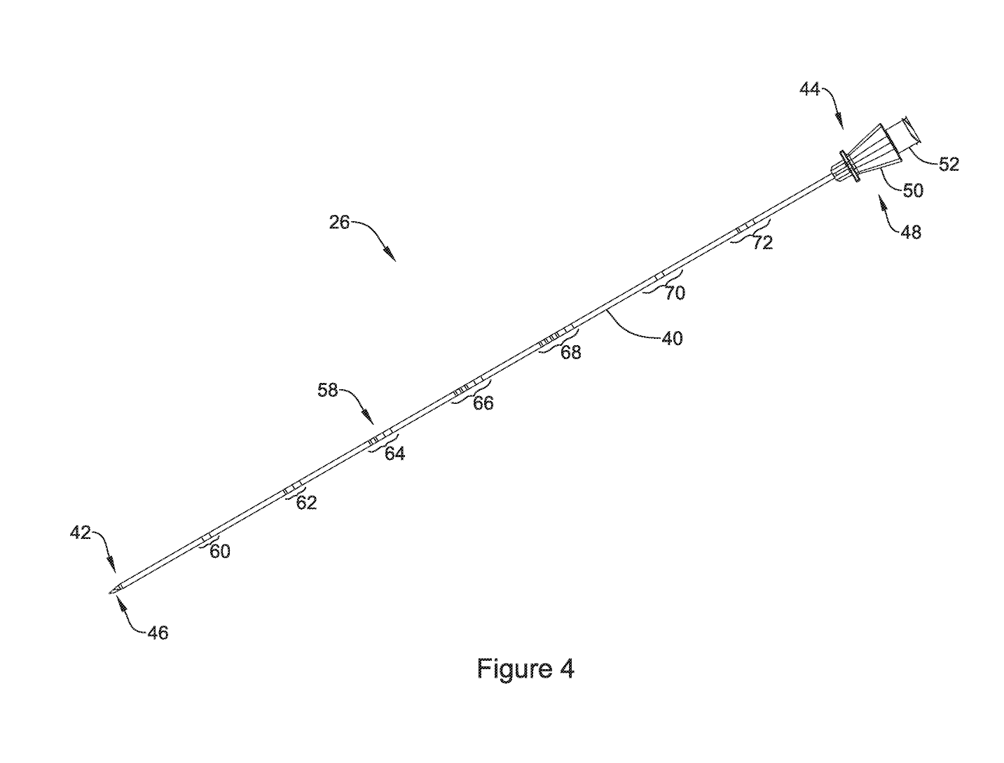

FIGS. 2 through 4 illustrate the primary components of a PCNL needle 22. In some cases, a PCNL needle 22 includes a stylet 24 (seen in FIG. 3) and a cannula 26 (seen in FIG. 4). The stylet 24 includes a stylet shaft 38 that extends between a distal end 28 and a proximal 30. In some cases, as illustrated, the stylet 24 includes a tapered portion 32 that is disposed at or near the distal end 28 of the stylet shaft 38. In some cases, the tapered portion 32 facilitates penetration of the skin during initial use of the PCNL needle 22. The stylet 24 may include a stylet hub 34 that is located at or near the proximal end 30 of the stylet shaft 38. The stylet hub 34 may be configured to be easily graspable and may be configured, as will be explained, to be releasably secured to a hub of the cannula 26, thereby locking the stylet 24 to the cannula 26 such that the assembly may be inserted into a patient as a unitary structure. In some cases, the stylet shaft 38 may be hollow. In some instances, particularly if the stylet 24 is removed prior to guidewire insertion, the stylet shaft 38 may instead be solid, for additional strength.

The cannula 26 includes a cannula shaft 40 that extends between a distal end 42 and a proximal end 44. In some cases, the cannula shaft 40 has a constant outer diameter all the way to the distal end 42 of the cannula shaft 40. In some cases, as shown, the cannula shaft 40 has a reduced diameter portion 46 disposed at the distal end 42 of the cannula shaft 40. The reduced diameter portion 46 may, in combination with the tapered portion 32 of the stylet shaft 38, facilitate insertion of the PCNL needle 22 into and through the patient's skin to the kidney 10 (FIG. 1). The cannula 26 may include a cannula hub 48 that is located at or near the proximal end 44 of the cannula shaft 40. In some cases, the cannula hub 48 includes a graspable portion 50 and a locking portion 52. In some cases, the graspable portion 50 may be configured to fit easily within an operator's fingers. The locking portion 52 may be configured to releasably secure the cannula hub 48 (and hence the cannula 26) relative to the stylet hub 34 (and hence the stylet 24).

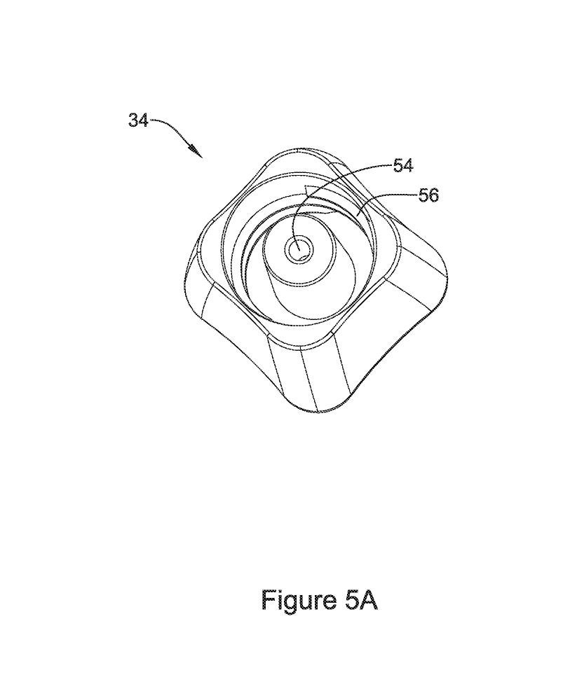

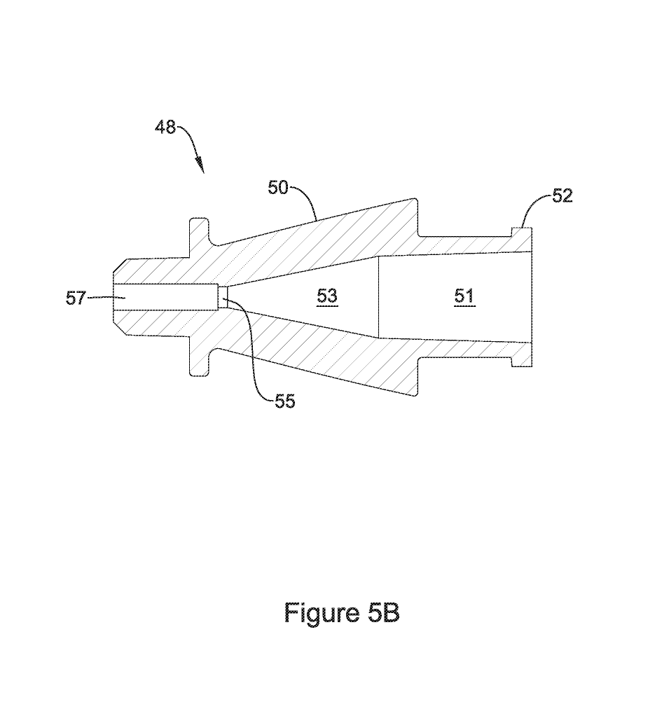

FIG. 5A provides a view of an interior of the stylet hub 34 in which the stylet shaft 38 has been removed for clarity. The stylet hub 34 can be seen as including a recess 54 that is configured to receive the stylet shaft 38. In some cases, the stylet hub 34 may be molded directly onto the stylet shaft 38. In some instances, the stylet hub 34 may be separately formed and subsequently secured in place on the stylet shaft 38. It can be seen that the interior of the stylet hub 34 includes a threaded portion 56. It will be appreciated that the threaded portion 56 of the stylet hub 34 may be configured to threadedly engage the locking portion 52 of the cannula hub 48.

FIG. 5B provides a cross-sectional view through the cannula hub 48, illustrating features of a guidewire lumen extending through the cannula hub 48. In some cases, as illustrated, the cannula hub 48 defines a first lumen portion 51 and a second lumen portion 53. In some instances, the first lumen portion 51 may be seen to have a slight taper, decreasing slightly in diameter moving proximal to distal. In some instances, the second lumen portion 53 has a more pronounced taper, decreasing in diameter moving proximal to distal. It will be appreciated that these tapers may facilitate insertion of a guidewire into the cannula hub 48. In some cases, the cannula hub 48 defines a constant diameter lumen portion 55 that may correspond to an inner diameter of the cannula shaft 40 (not illustrated). The cannula hub 48 may include a region 57 that is configured to accommodate the cannula shaft 40.

Returning briefly to FIG. 2, in some cases, the cannula hub 48 may include a first alignment marker 74 and the stylet hub 34 may include a second alignment marker 76. In some indications, the first alignment marker 74 and the second alignment marker 76 may, in combination, provide a visual indication whether the stylet hub 34 is fully engaged with the cannula hub 48. For example, in some cases, as illustrated, the first alignment marker 74 may align with the second alignment marker 76 when the cannula hub 48 is fully and correctly secured to the stylet hub 34. This tells the physician that the stylet 22 and the cannula 26 are secured together and may be handled as a unitary structure. In some cases, the first alignment marker 74 and/or the second alignment marker 76 may be printed or engraved in place.

Returning to FIG. 4, in some cases the cannula shaft 40 includes a depth guide 58 that in some cases provides an indication of insertion depth when the PCNL needle 22 is being inserted into a patient. A physician may, for example, have an idea as to how far into the patient they wish to insert the PCNL needle 22 in order to reach a desired location within the kidney 10 (FIG. 1), based upon one or more imaging techniques. The depth guide 58 may for example be printed onto the cannula shaft 40. In some cases, the depth guide 58 may be engraved into the surface of the cannula shaft 40. The depth guide 58 may take any form. In some cases, the depth guide 58 may essentially be a ruler, with markings every centimeter, for example.

In some cases, the depth guide 58 may include a plurality of markings that indicate relative position of each marking or group of markings on the cannula shaft 40. For example, and as illustrated and moving from distal to proximal, the depth guide 58 may include a first marking 60 including a single wide band, a second marking 62 including a single narrow band and a single wide band, a third marking 64 including two narrow bands and one wide band, a fourth marking 66 including three narrow bands and one wide band and a fifth marking 68 including four narrow bands and one wide band. In some instances, the depth guide 58 may include a sixth marking 70 including a single wide band and a seventh marking 72 including a single narrow band and a single wide band. The sixth marking 70 and the seventh marking 72 may, for example, be included to provide a warning to the physician that they are approaching an insertion depth that may be too deep to remain at a desired target location within the kidney 10 (FIG. 1).

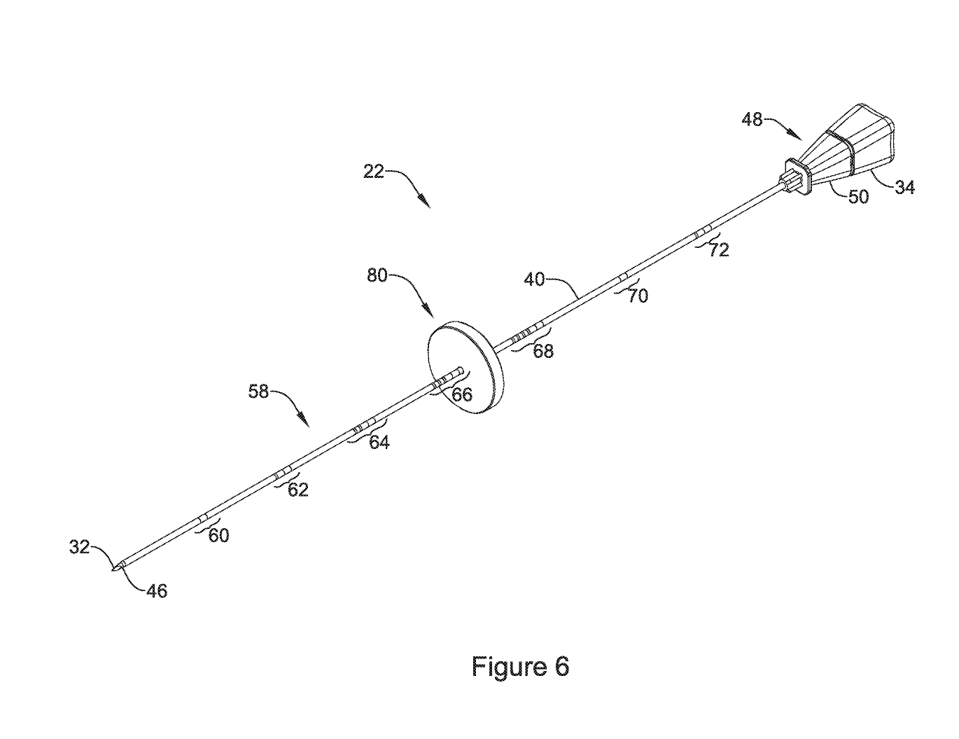

FIG. 6 is a perspective view of the PCNL needle 22, much as shown in FIG. 2, with the addition of an adjustable depth stop 80. In some cases, the adjustable depth stop 80 may be releasably securable to the cannula shaft 40 at a desired position relative to the depth guide 58. In some cases, the adjustable depth guide 80 is capable of being manipulated between an adjustment configuration in which the adjustable depth guide 80 is moveable relative to the cannula shaft 40 and a secured configuration in which the adjustable depth guide 80 is secured relative to the cannula shaft 40. The adjustable depth guide 80, which may also be considered as being an adjustable slider may be formed of any of a variety of different polymeric materials. In some cases, the adjustable depth guide 80 may be molded or otherwise formed of a polymer such as nylon or polypropylene.

In some cases, at least a portion of the adjustable depth guide 80 may be radiopaque, so that the adjustable depth guide 80 may be visible during fluoroscopy or other imaging techniques. In some cases, for example, during the PCNL process, the operator may move the PCNL needle 22 between several different angular orientations in order to visualize the angle and depth required to reach a desired position (depth and angle) within the kidney 10. In some cases, the PCNL needle 22 may be moved between a bulls eye orientation (C-arm at 30 degrees) that provides a direction but not a distance, and a top view orientation (C-arm at 90 degrees) that provides the distance.

With the C-arm at the 90 degree orientation, the adjustable depth stop 80, which is visible under fluoroscopy, can be positioned relative to the cannula shaft 40 to mark an insertion depth that indicates a location of the kidney stone relative to the skin. Once the depth has been marked, the C-arm can be inclined to the 30 degree (bulls eye) orientation, which makes the PCNL needle 22 coaxial with the calyx and the kidney stone.

In some cases, a radiopaque material may be admixed with the polymer forming the adjustable depth guide 80, for example. A radiopaque ink may be printed on at least a portion of the adjustable depth guide 80. In some cases, the adjustable depth guide 80 may include a metallic ring or other structure to provide radiopacity. The adjustable depth guide 80 may take a variety of different forms, as will be illustrated with respect to subsequent Figures.

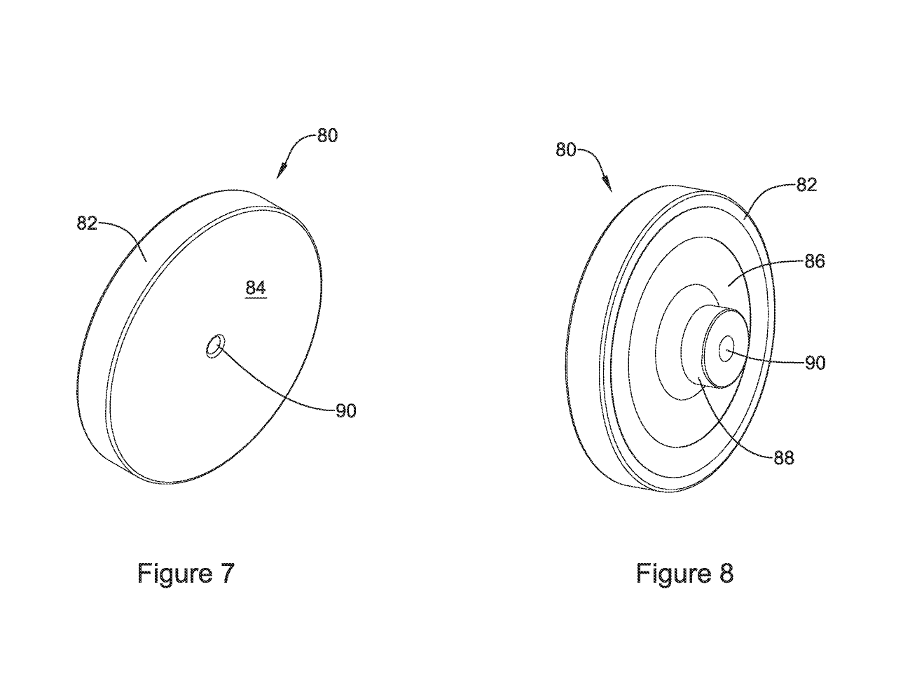

FIGS. 7 and 8 provide additional details regarding the adjustable depth guide 80. The adjustable depth guide 80 includes a generally annular body 82 having a first generally planar surface 84 and a second generally planar surface 86. In some cases, the second generally planar surface 86 includes a raised portion 88, with an aperture 90 extending through the generally annular body 82 and through the raised portion 88. The aperture 90 may, for example, be configured to provide a frictional fit with the cannula shaft 40 such that the annular body 82 may be forceably slid relative to the cannula shaft 40 upon application of a directed force, but holds the annular body 82 in position on the cannula shaft 40 when the directed force is not being applied.

In some cases, the raised portion 88 may provide an easily graspable portion of the adjustable depth stop 80 that a user may grasp, such as between their index finger and thumb, in order to adjust the position of the adjustable depth stop 80. In some cases, while the raised portion 88 is illustrated as being at least substantially cylindrical, the raised portion 88 may have a square or substantially square cross-sectional profile that may be easier to grasp. In some cases, the raised portion 88 functions to lengthen the effective length of the aperture 90 in order to increase the frictional fit. In some cases, the raised portion 88 may help with a more accurate measurement, particularly in the case of an angled delivery. Because the raised portion 88 has a smaller width than the annular body 82, the raised portion 88 may contact the patient's skin at the desired depth more accurately. Without the narrower raised portion 88, it may be possible to underestimate the distance and/or depth because one side of the annular body 82 would contact the skin ahead of the desired, measured depth.

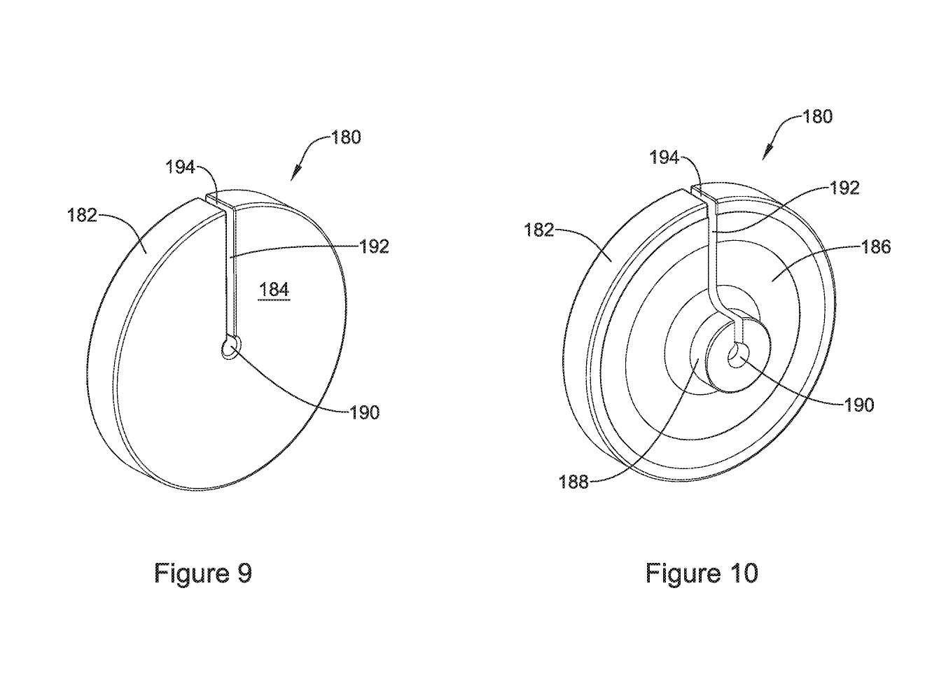

FIGS. 9 and 10 provide several views of an adjustable depth guide 180. The adjustable depth guide 180 includes a generally annular body 182 having a first generally planar surface 184 and a second generally planar surface 186. In some cases, the second generally planar surface 186 includes a raised portion 188, with an aperture 190 extending through the generally annular body 182 and through the raised portion 188. The aperture 190 may, for example, be configured to provide a frictional fit with the cannula shaft 40. In some cases, as illustrated, the annular body 182 may include a channel 192 that extends from a perimeter 194 of the annular body 182 to the aperture 190. In some cases, the channel 192 has a width that is less than a width of the aperture 190. In some instances, the channel 192 facilitates bending or otherwise temporarily deforming the annular body 182 in order to facilitate initially placing the adjustable depth guide 180 on the cannula shaft 40 and/or to adjust the position of the adjustable depth guide 180 relative to the depth guide 58 on the cannula shaft 40.

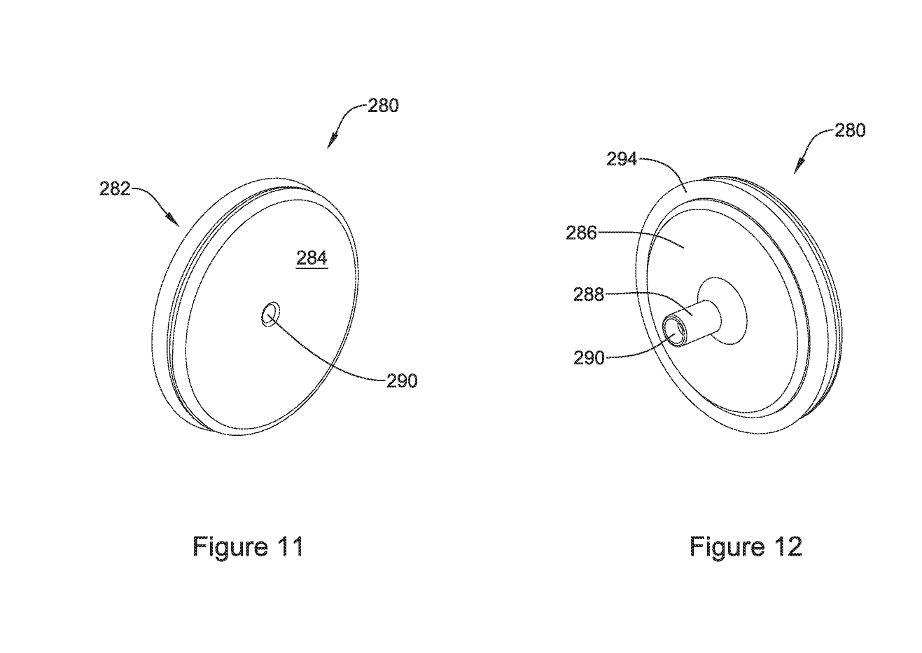

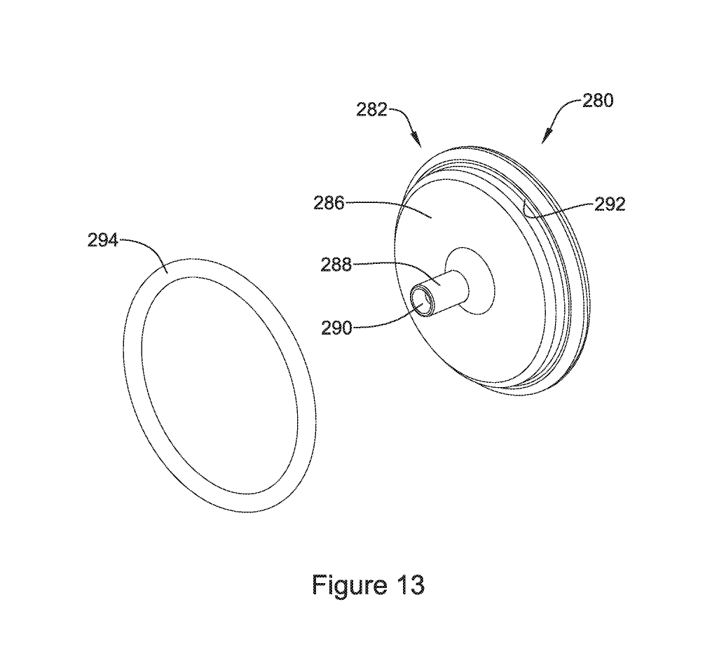

FIGS. 11 through 13 provide several views of an adjustable depth guide 280. The adjustable depth guide 280 includes a generally annular body 282 having a first generally planar surface 284 and a second generally planar surface 286. In some cases, the second generally planar surface 286 includes a raised portion 288, with an aperture 290 extending through the generally annular body 282 and through the raised portion 288. The aperture 290 may, for example, be configured to provide a frictional fit with the cannula shaft 40. In some cases, the second generally planar surface 286 includes an annular recess 292 that is configured to accommodate a radiopaque ring 294. In some cases, the radiopaque ring 294 may be molded into the adjustable depth guide 280 via a co-molding process. In some cases, the radiopaque ring may be formed of a radiopaque metal such as steel, aluminum, tungsten, platinum, gold, silver, nitinol or a cobalt alloy such as cobalt chromium. Other metals may also be used. In some cases, rather than a metal ring, the radiopaque ring 294 may instead be formed via printing on the adjustable depth guide 280 using a radiopaque ink.

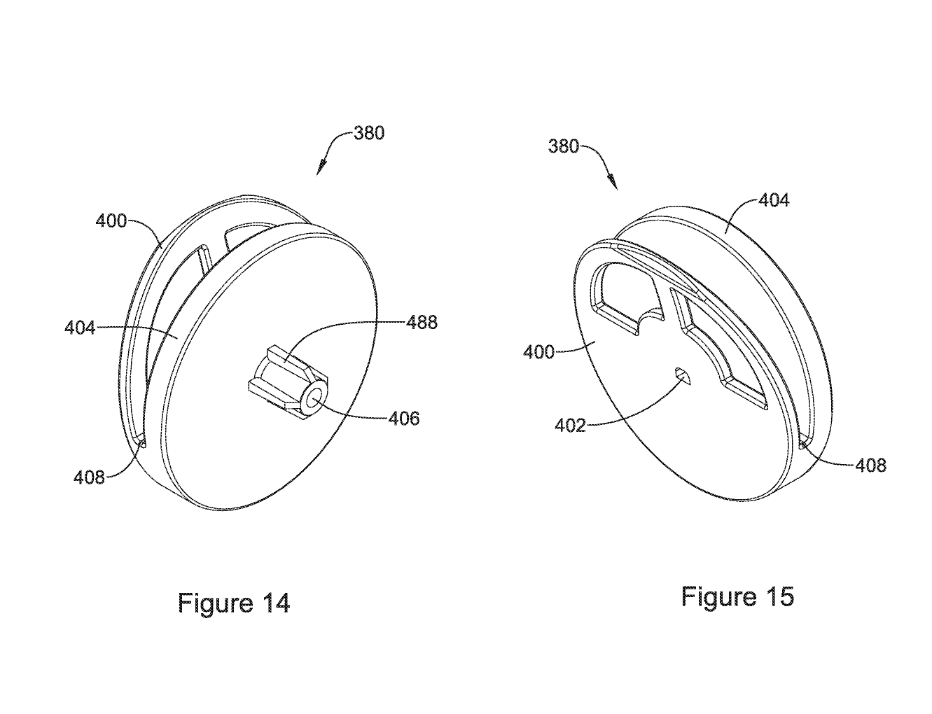

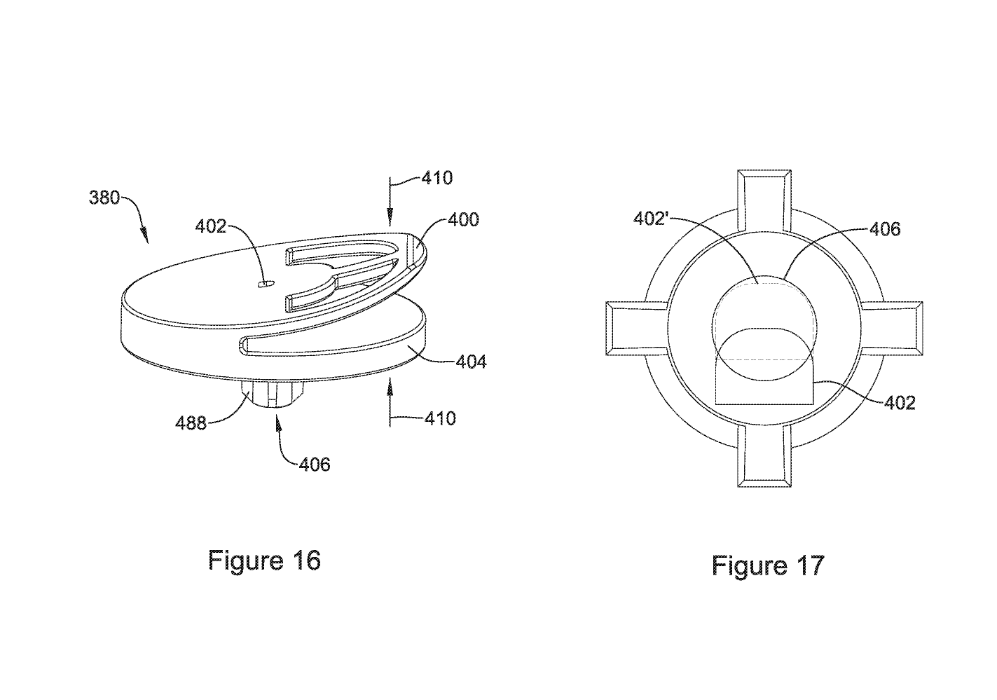

FIGS. 14 through 16 provide several views of an adjustable depth guide 380 and FIG. 17 schematically shows the alignment between a first aperture and a second aperture, as will be described. In some cases, the adjustable depth guide 380 may be considered as including a first leaf 400 having a first aperture 402 that is configured to accommodate the cannula shaft 80 and a second leaf 404 having a second aperture 406 that is configured to accommodate the cannula shaft 80. In some cases, the second aperture 406 extends through a raised portion 488. The first leaf 400 and the second leaf 402 may, for example, be joined together via a living hinge 408.

In some cases, as shown for example in FIG. 17, the first leaf 400 and the second leaf 404 may, in combination, be biased to a position in which the first aperture 402 is at least partially misaligned with the second aperture 406. As a result, in the relaxed configuration shown in FIGS. 14 through 16, the adjustable depth guide 380 is secured in place on the cannula shaft 80. By squeezing the first leaf 400 and the second leaf 404 together, as indicated by arrows 410, the first aperture 402 may be moved into alignment with the second aperture 406, thereby permitting the adjustable depth guide 380 to be moved relative to the cannula shaft 80. When the first leaf 400 and the second leaf 404 are squeezed together, the first aperture 402 and the second aperture 406 align, as indicated in FIG. 17 by the second position of the first aperture 402, shown in phantom and labeled as 402′.

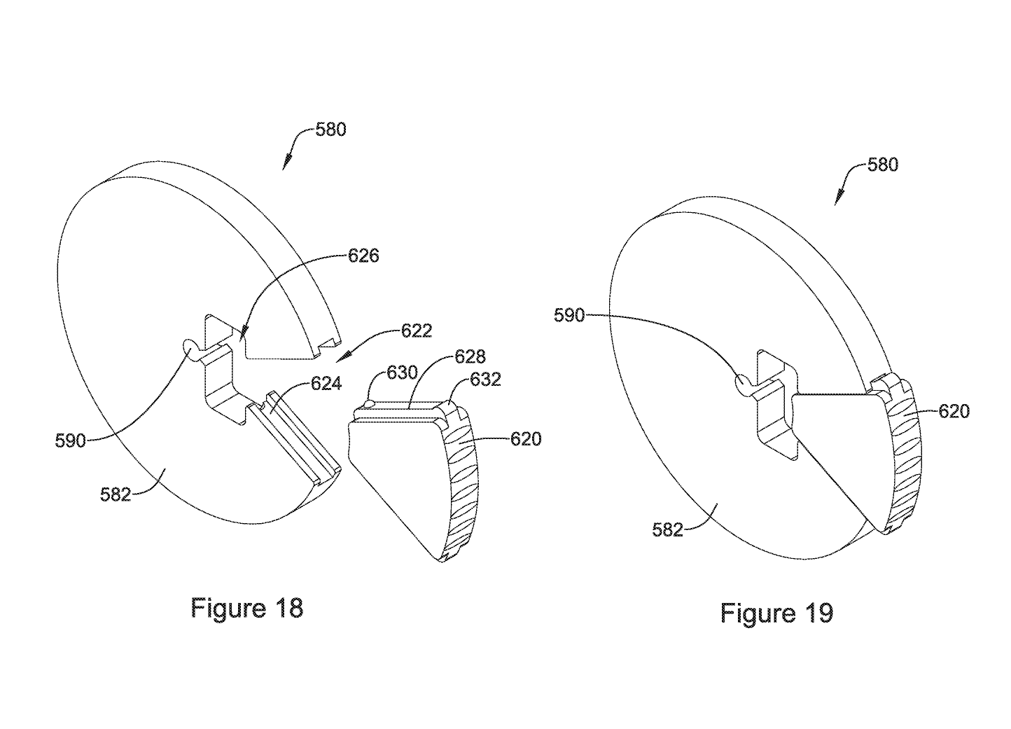

FIGS. 18 and 19 provide several views of an adjustable depth stop 580. The adjustable depth stop 580 includes a generally annular body 582 and a removable wedge 620. An aperture 590 extends through the generally annular body 582 and may be configured to accommodate the cannula shaft 80 extending therethrough. In some cases, the aperture 590 may have a relaxed configuration (as shown in FIG. 18) in which the aperture 590 frictionally engages the cannula shaft 80. The annular body 580 defines a wedge shaped recess 622 that is sized to accommodate the removable wedge 620 as well as an additional recess 624 extending between the wedge shaped recess 622 and the aperture 590. When the removable wedge 620 is inserted into the wedge shaped recess 622 and urged towards the aperture 590, the annular body 580 may be deformed sufficiently to increase an effective diameter of the aperture 590, thereby permitting the cannula shaft 80 to slide relative to the aperture 590. In some cases, the position of the removable wedge 620 relative to the wedge-shaped recess 622, as shown for example in FIG. 19, may represent a position in which the effective diameter of the aperture 590 is not being materially impacted by the removable wedge 620.

In some cases, the wedge-shaped recess 622 include a channel 624 disposed on a side of the wedge-shaped recess 622 to accommodate a corresponding raised portion 628 on a side of the removable wedge 620. The raised portion 628 may fit into the channel 624 such that one functions as a track, guiding the other. In some cases, the wedge-shaped recess 622 may include a channel 624 on each side of the wedge-shaped recess 622 and the removable wedge 620 may include a raised portion 628 on each side of the removable wedge 620. In some cases, the raised portion 628 may include one or more retention features such as a raised feature 630 on either side. A flared portion 632 may limit how far the removable wedge 620 may be forced into the wedge-shaped recess 622.

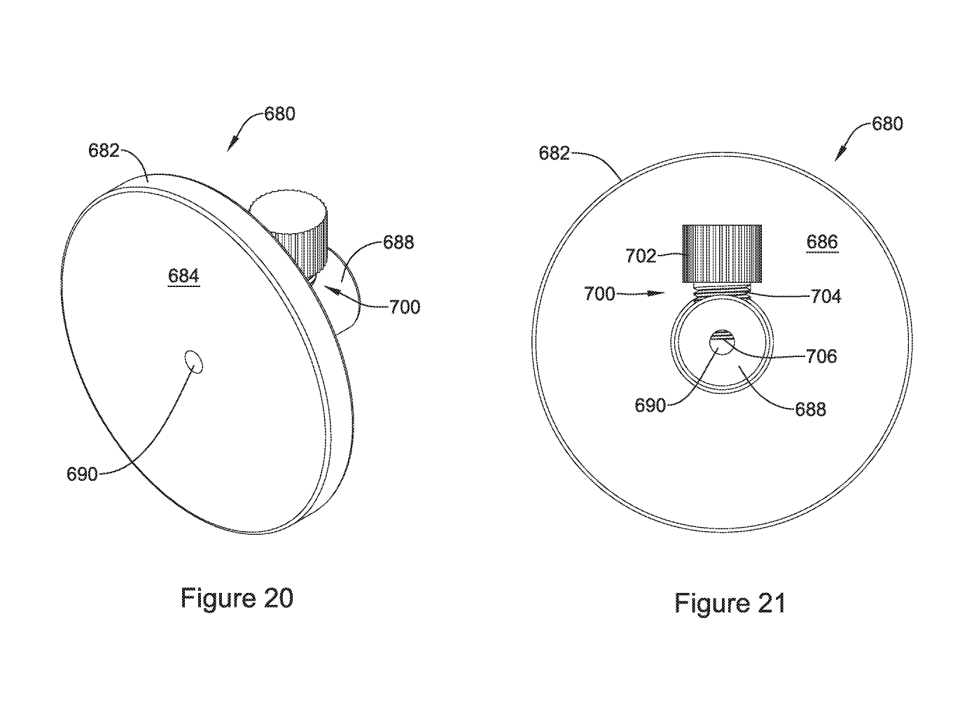

FIGS. 20 and 21 provide several views of an adjustable depth guide 680. The adjustable depth guide 680 includes a generally annular body 682 having a first generally planar surface 684 and a second generally planar surface 686. In some cases, the second generally planar surface 686 includes a raised portion 688, with an aperture 690 extending through the generally annular body 682 and through the raised portion 688. The aperture 690 may, for example, be configured to easily accommodate the cannula shaft 40 such that the annular body 682 may easily be slid relative to the cannula shaft 40. In some cases, the raised portion 688 accommodates a fastener 700 having a graspable portion 702 and a threaded portion 704 that engages a corresponding threaded aperture within the raised portion 688. The fastener 700 includes a bottom surface 706 that may be moved into contact with the cannula shaft 40 in order to secure the adjustable depth guide 680 in place relative to the cannula shaft 40 and/or moved away from the cannula shaft 40 to permit the adjustable depth guide 680 to move relative to the cannula shaft 40.

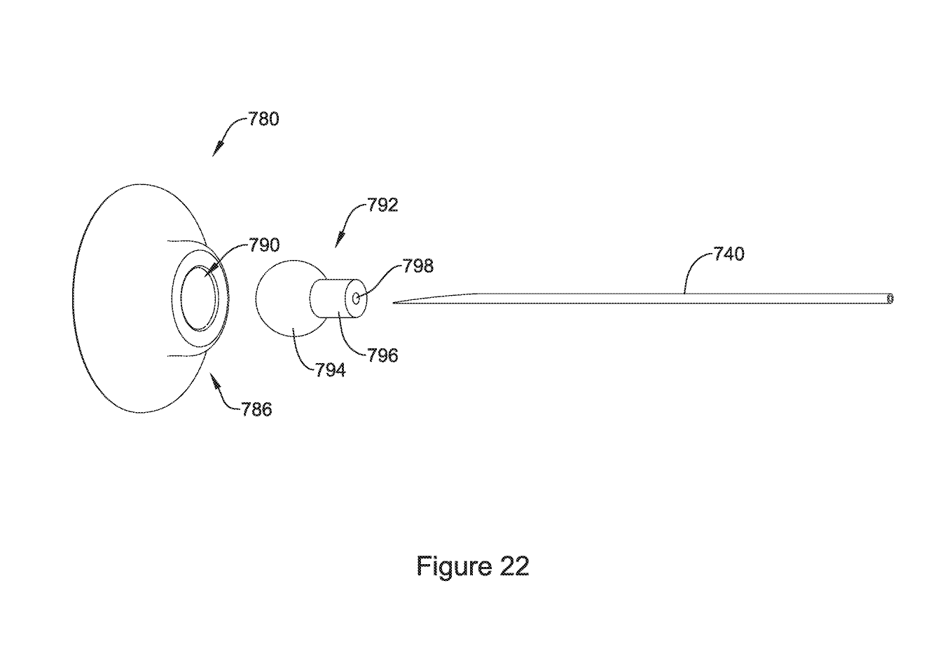

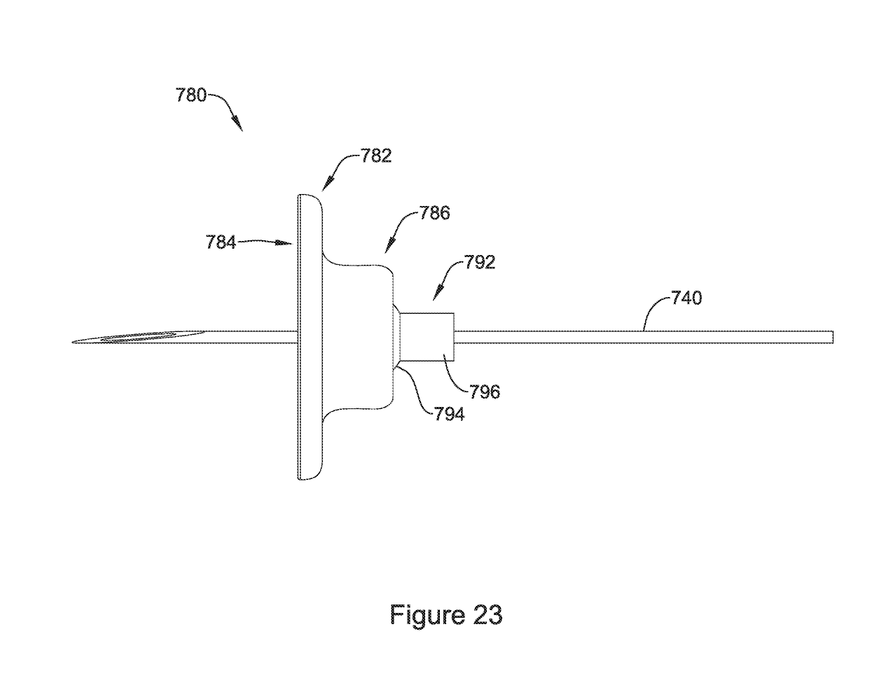

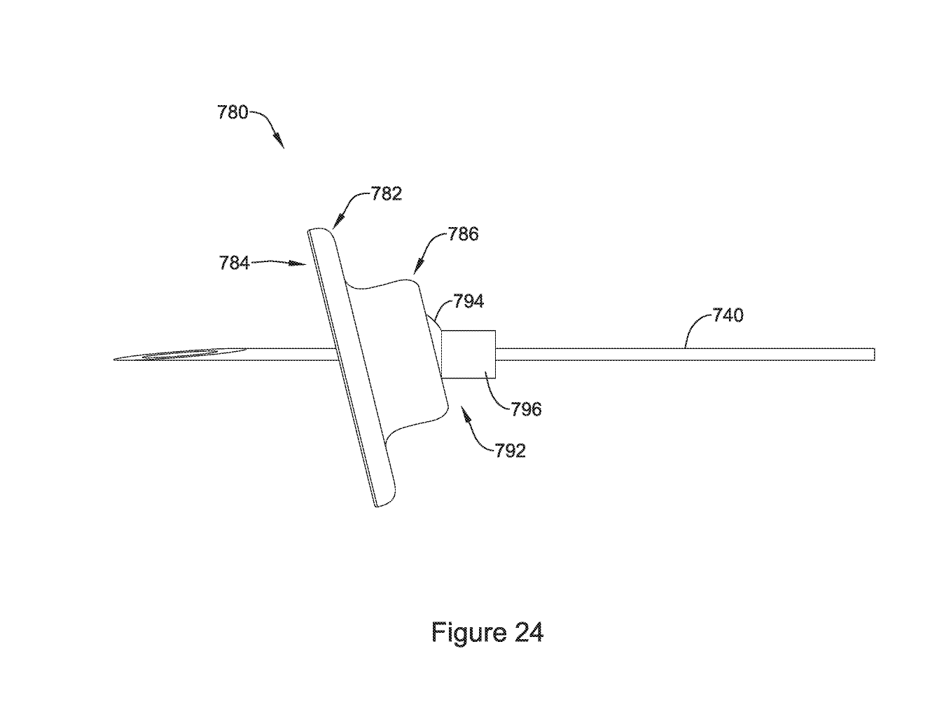

FIGS. 22 through 24 provides several views of an adjustable depth guide 780 that is shown disposed on a needle 740. The adjustable depth guide 780 includes a generally annular body 782 having a generally planar surface 784 on one side of the generally annular body 782 and a protruding portion 786 on an opposing side of the generally annular body 782. The protruding portion 786 includes a void 790 that may be configured to accommodate a pivoting member 792. In some cases, the pivoting member 792 is formed of a material that is soft enough to be able to be popped into position within the void 790. In some cases, the pivoting member 792 includes a spherical portion 794 that is capable of rotating in any direction relative to the void 790 as well as a cylindrical portion 796. The pivoting member 792 includes an aperture 798 that extends axially through the cylindrical portion 796 as well as the spherical portion 794 in order to accommodate the needle 740. By comparing FIG. 23 to FIG. 24, it can be seen that the pivoting member 792 is able to rotate relative to the generally annular body 782 and thus can accommodate various angles between the adjustable depth guide 780 and the needle 740.

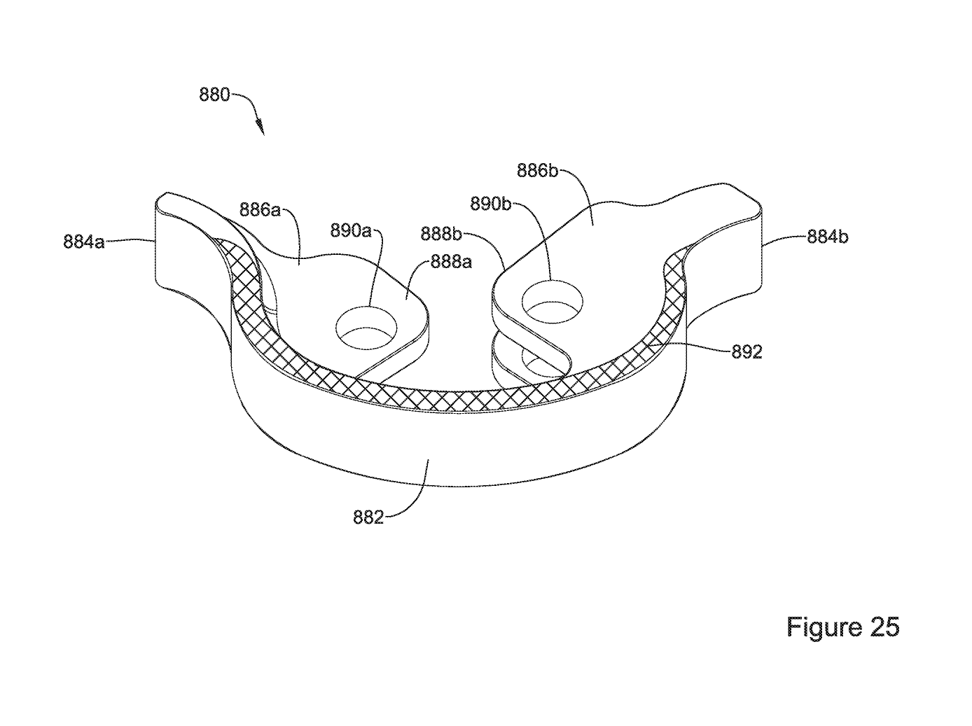

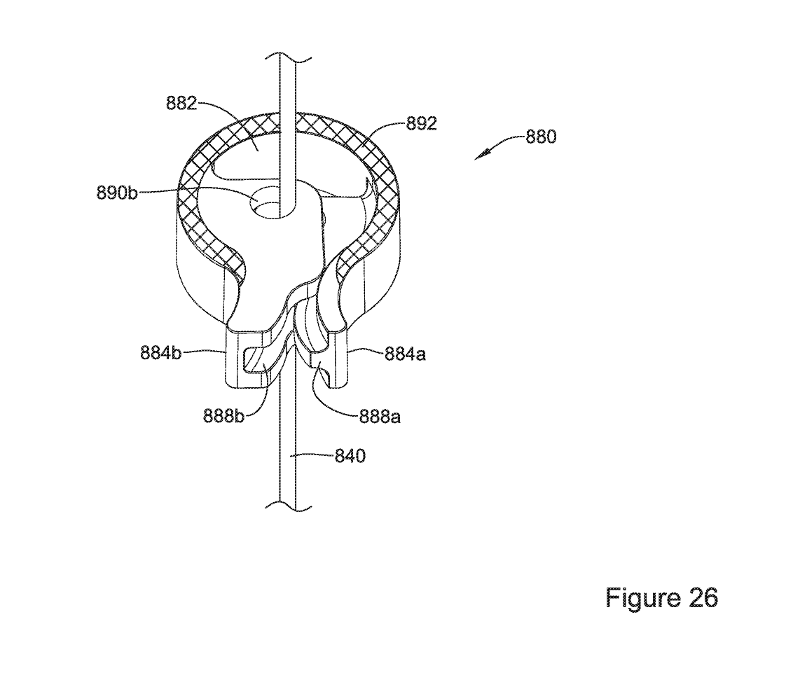

FIG. 25 shows an adjustable depth stop 880 and FIG. 26 shows the adjustable depth stop 880 disposed on a needle 840. The adjustable depth stop 880 includes a main body 882 including a first graspable end portion 884a and a second graspable end portion 884b. In some cases, the main body 882 includes a first section 886a that is relatively closer to the first graspable end portion 884a and a second section 886b that is relatively closer to the second graspable end portion 884b. In some cases, as illustrated, the first section 886a may define a planar portion 888a that may be configured to fit into a slotted portion 888b. The main body 882 may be formed of a sufficiently flexible material, such as but not limited to nylon or polypropylene, such that the first graspable end portion 884a and the second graspable end portion 884b may be squeezed together and the planar portion 888a may slide into the slotted portion 888b. In some cases, the adjustable depth stop 880 may include a radiopaque feature 892 that facilitates fluoroscopic imaging of the adjustable depth stop 880. In some cases, for example, the radiopaque feature 892 may be a printed radiopaque ink.

A first aperture 892a may be formed within the planar portion 888a and a second aperture 892b may be formed within the slotted portion 888b. As can be seen by comparing FIG. 25 with FIG. 26, as the first graspable end portion 884a and the second graspable end portion 884b are squeezed together, and the planar portion 888a slides into the slotted portion 888b, the first aperture 892a will align with the second aperture 892b. As a result, and as shown in FIG. 26, the adjustable depth stop 880 may be slid over the needle 840. Once the adjustable depth stop 880 is in a desired location, letting go of the first graspable end portion 884a and the second graspable portion 884b will cause the planar portion 888a to move slightly away from the slotted portion 888b, limited by interference between the needle 840 and the first and second apertures 892a, 892b. As a result, the adjustable depth stop 880 is securable at a desired location on the needle 840, and can be moved or otherwise further adjusted as desired.

The PCNL needle 22, including the stylet 24 (and stylet shaft 38) and the cannula 26 (and cannula shaft 40) or components thereof may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

Some components of the PCNL needle 22, such as the stylet hub 34 and the cannula hub 48, and the adjustable depth guide 80, 180, 280, 380, 480, 580, 680, 780 and 880 may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

### ADDITIONAL EXAMPLES

### Example 1

A percutaneous nephrolithotomy access needle comprising:
a cannula including a shaft defining a cannula lumen extending from a proximal end of the cannula to a distal end of the cannula, the shaft having an outer surface;
a depth guide disposed on the outer surface of the cannula shaft, the depth guide configured to provide an indication of insertion depth;
a cannula hub coupled to the proximal end of the cannula;
a stylet disposable within the cannula lumen and extending from a proximal end of the stylet to a distal end of the stylet, the stylet including a tapered point at the distal end of the stylet;
a stylet hub coupled to a proximal end of the stylet, the stylet hub configured to be releasably securable to the cannula hub; and
a first alignment marker disposed on the cannula hub and a second alignment marker disposed on the stylet hub, the first alignment marker and the second alignment marker positioned to provide a visual indication that the stylet hub is aligned with the cannula hub.

### Example 2

The percutaneous nephrolithotomy access needle of Example 1, further comprising a wire insertion lumen extending through the cannula hub.

### Example 3

The percutaneous nephrolithotomy access needle of Example 2, wherein the wire insertion lumen is tapered, with a maximum diameter near the proximal end of the cannula hub and a minimum diameter at a position near where the cannula hub is coupled to the cannula shaft.

### Example 4

The percutaneous nephrolithotomy access needle of Example 3, wherein the minimum diameter of the tapered wire insertion lumen is about the same as an inner diameter of the cannula shaft.

### Example 5

The percutaneous nephrolithotomy access needle of Example 1, wherein the stylet has a length sufficient to permit the tapered point of the stylet to extend distally from the distal end of the cannula when the stylet hub is secured to the cannula hub.

### Example 6

The percutaneous nephrolithotomy access needle of Example 1, wherein the cannula hub has an outer surface that is configured to be easily graspable.

### Example 7

The percutaneous nephrolithotomy access needle of Example 1, wherein the cannula hub has a rectilinear profile.

### Example 8

The percutaneous nephrolithotomy access needle of Example 1, wherein the stylet hub has a threaded engagement with the cannula hub.

### Example 9

The percutaneous nephrolithotomy access needle of Example 1, wherein the cannula hub is molded onto the cannula.

### Example 10

A percutaneous nephrolithotomy access needle comprising:
a cannula including a cannula lumen extending through the cannula, the cannula having an outer surface;
a cannula hub coupled to a proximal end of the cannula, a first alignment marker disposed on the cannula hub;
a stylet disposed within the cannula lumen, the stylet including a tapered distal point extending past a distal end of the cannula;
a stylet hub coupled to a proximal end of the stylet, the stylet hub configured to be releasably securable to the cannula hub, a second alignment marker disposed on the stylet hub; and
a tapered wire insertion lumen extending through the cannula hub;
wherein an alignment between the first alignment marker and the second alignment marker indicates whether the stylet hub is aligned with the cannula hub.

### Example 11

The percutaneous nephrolithotomy access needle of Example 10, further comprising a depth guide disposed on the outer surface of the cannula, the depth guide providing an indication of insertion depth.

### Example 12

The percutaneous nephrolithotomy access needle of Example 10, wherein the cannula hub has an outer surface that is configured to be easily graspable.

### Example 13

The percutaneous nephrolithotomy access needle of Example 10, wherein the cannula hub has a rectilinear profile.

### Example 14

The percutaneous nephrolithotomy access needle of Example 10, wherein the stylet and the cannula are operably coupled together when the stylet hub is secured to the cannula hub.

### Example 15

The percutaneous nephrolithotomy access needle of Example 10, wherein the stylet hub has a threaded engagement with the cannula hub.

### Example 16

A kit for providing percutaneous access to a patient's renal pelvis, the kit comprising:
a needle guide including a cylindrical body configured to be easily grasped, the needle guide further comprising an enlarged portion configured to contact the patient's skin and provide a flattened skin portion through which to access the renal pelvis and a needle lumen; and
a percutaneous nephrolithotomy access needle configured to be extend through the needle lumen, the percutaneous nephrolithotomy access needle comprising:
   a cannula including a shaft defining a cannula lumen extending from a proximal end of the cannula to a distal end of the cannula, the shaft having an outer surface;
   a depth guide disposed on the outer surface of the cannula shaft, the depth guide configured to provide an indication of insertion depth;
   a cannula hub coupled to the proximal end of the cannula; and
   a stylet disposable within the cannula lumen and extending from a proximal end of the stylet to a distal end of the stylet, the stylet including a tapered point at the distal end of the stylet;
a stylet hub coupled to a proximal end of the stylet, the stylet hub configured to be releasably securable to the cannula hub; and
a first alignment marker disposed on the cannula hub and a second alignment marker disposed on the stylet hub, the first alignment marker and the second alignment marker positioned to provide a visual indication that the stylet hub is aligned with the cannula hub.

### Example 17

The kit for providing percutaneous access to a patient's renal pelvis of Example 16, wherein the stylet has a length sufficient to permit the tapered point of the stylet to extend distally from the distal end of the cannula when the stylet hub is secured to the cannula hub.

### Example 18

The kit for providing percutaneous access to a patient's renal pelvis of Example 16, wherein the cannula hub has a rectilinear profile.

### Example 19

The kit for providing percutaneous access to a patient's renal pelvis of Example 16, wherein the stylet hub has a threaded engagement with the cannula hub.

### Example 20

The kit for providing percutaneous access to a patient's renal pelvis of Example 16, wherein the cannula hub is molded onto the cannula.

### Example 21

An adjustable depth stop configured for use in combination with a PCNL needle, the adjustable depth stop comprising:
an annular body having an outer perimeter;
an aperture extending through the annular body at a central location thereof, the aperture sized to provide a frictional fit with a PCNL needle shaft;
a wedge shaped recess formed within the annular body, the wedge shaped recess extending into the annular body from the outer perimeter;
a secondary recess disposed between the wedge shaped recess and the aperture, the secondary recess forming a portion of a perimeter wall of the aperture;
a removable wedge disposable within the wedge shaped recess, the removable wedge movable between a relaxed configuration in which the removable wedge does not impact a diameter of the aperture and an advanced configuration in which the removable wedge has been urged towards the aperture, and the aperture is opened slightly in order to permit the adjustable depth stop to be moved relative to the PCNL needle shaft.

### Example 22

An adjustable depth stop configured for use in combination with a PCNL needle, the adjustable depth stop comprising:
an annular body;
a needle aperture extending through the annular body, the needle aperture sized to provide a frictional fit with a PCNL needle shaft;
a raised portion extending outwardly from a side of the annular body, the needle aperture extending through the raised portion;
a threaded aperture extending into the raised portion perpendicularly to the needle aperture; and
a threaded fastener disposed within the threaded aperture, the threaded fastener movable between a position in which the threaded fastener frictionally engages the PCNL needle shaft and limits movement of the adjustable depth stop relative to the PCNL needle shaft and a position in which the threaded fastener does not frictionally engage the PCNL needle shaft.

### Example 23

An adjustable depth stop configured for use in combination with a PCNL needle, the adjustable depth stop comprising:
an annular body;
a protruding portion extending outwardly from the annular body, the protruding portion defining a pivot member void therein;
a pivot member rotatably disposable within the pivot member void, the pivot member including a spherical portion configured to rotate within the pivot member void and a cylindrical portion extending outwardly from the spherical portion;
an aperture extending through the annular body at a central location thereof, the aperture sized to provide a frictional fit with a PCNL needle shaft;

### Example 24

An adjustable depth stop configured for use in combination with a PCNL needle, the adjustable depth stop comprising:
a main body including a first section defining a planar portion and a second section defining a slotted portion;
the main body including a first graspable end portion disposed proximate the first section;
the main body including a second graspable end portion disposed proximate the second section; a first end of the main body;
a first aperture formed within the first section;
a second aperture formed within the second section; and
a radiopaque feature disposed on the main body;
wherein moving the first graspable end portion towards the second graspable end portion causes the planar portion of the first section to slide into the slotted portion of the second section such that the first and second apertures align to accommodate a needle therethrough.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous nephrolithotomy (PCNL) needle, comprising:
a cannula including a shaft defining a cannula lumen extending through the shaft and a cannula hub coupled to a proximal end of the cannula;
a depth guide disposed on an outer surface of the cannula shaft, the depth guide configured to provide an indication of insertion depth;
a stylet disposable within the cannula lumen, the stylet including a tapered point at a distal end of the stylet and a stylet hub coupled to a proximal end of the stylet, the stylet hub configured to be releasably securable to the cannula hub; and
an adjustable depth stop releasably securable to the cannula shaft at a desired position relative to the depth guide, the adjustable depth guide capable of being manipulated between an adjustment configuration in which the adjustable depth guide is moveable relative to the cannula shaft and a secured configuration in which the adjustable depth guide is secured relative to the cannula shaft.

2. The PCNL needle of claim 1, further comprising a first alignment marker disposed on the cannula hub and a second alignment marker disposed on the stylet hub, the first alignment marker and the second alignment marker positioned to provide a visual indication that the stylet hub is aligned with the cannula hub.

3. The PCNL needle of claim 1, further comprising a tapered guidewire lumen extending through the cannula hub.

4. The PCNL needle of claim 1, wherein the stylet has a length sufficient to permit the tapered point of the stylet to extend distally from a distal end of the cannula when the stylet hub is secured to the cannula hub.

5. The PCNL needle of claim 1, wherein the stylet hub is configured to threadedly engage with the cannula hub.

6. The PCNL needle of claim 1, wherein the adjustable depth stop comprises a radiopaque component.

7. The PCNL needle of claim 6, wherein the radiopaque component comprises a radiopaque ring disposed on the adjustable depth stop.

8. The PCNL needle of claim 6, wherein the radiopaque component comprises a radiopaque material admixed within a polymer forming the adjustable depth stop.

9. The PCNL needle of claim 6, wherein the radiopaque component comprises a radiopaque ink printed onto one or more surfaces of the annular body.

10. The PCNL needle of claim 1, wherein the adjustable depth stop comprises an annular body with an aperture extending through the annular body, the aperture configured to accommodate the cannula therethrough.

11. The PCNL needle of claim 10, wherein the aperture extending through the annular body is dimensioned to provide a frictional fit with the cannula shaft, such that the annular body may be forceably slid relative to the cannula shaft, but the aperture holds the annular body in place in the absence of force applied to the annular body.

12. The PCNL needle of claim 11, further comprising a slot formed in the annular body, extending outward from the aperture to a perimeter of the annular body.

13. The PCNL needle of claim 10, wherein the annular body further comprises a cylindrical portion extending concentrically with the cannula shaft, the cylindrical portion including a threaded securement aperture extending through the cylindrical portion orthogonally to the cannula shaft, the adjustable depth stop further comprising a threaded fastener engaged within the threaded securement aperture such that the threaded fastener is capable of being moved into contact with the cannula shaft in order to secure the adjustable depth stop relative to the cannula shaft.

14. The PCNL needle of claim 1, wherein the adjustable depth stop includes a first leaf having a first aperture sized to accommodate the cannula shaft and a second leaf having a second aperture sized to accommodate the cannula shaft, the first leaf and the second leaf joined via a living hinge.

15. The PCNL needle of claim 14, wherein:
the first leaf is biased to a position relative to the second leaf such that the first aperture is at least partially misaligned with the second aperture in order to secure the adjustable depth stop relative to the cannula shaft; and wherein
the first leaf and the second leaf are capable of being squeezed together to move the first aperture into alignment with the second aperture such that the adjustable depth stop may be moved relative to the cannula shaft.

16. A percutaneous nephrolithotomy (PCNL) access assembly, comprising:
an access needle comprising:
a cannula including a hub and a shaft extending from the hub;
a depth guide disposed on an outer surface of the cannula shaft in order to provide an indication of insertion depth;
a stylet including a tapered point at a distal end of the stylet and a stylet hub coupled to a proximal end of the stylet, the stylet hub configured to be releasably securable to the cannula hub with the stylet extending through the cannula; and
an adjustable slider releasably securable to the cannula shaft at a desired position relative to the depth guide, the adjustable slider capable of an adjustment configuration in which the adjustable slider is moveable relative to the cannula shaft and a secured configuration in which the adjustable slider is secured relative to the cannula shaft.

17. The PCNL access assembly of claim 16, wherein the adjustable slider comprises an annular body with an aperture extending through the annular body, the aperture dimensioned to provide a frictional fit with the cannula shaft, such that the annular body may be forceably slid relative to the cannula shaft, but the aperture holds the annular body in place in the absence of force applied to the annular body.

18. The PCNL access assembly of claim 16, wherein the annular body further comprises a cylindrical portion extending concentrically with the cannula shaft, the cylindrical portion including a threaded securement aperture extending through the cylindrical portion orthogonally to the cannula shaft, the adjustable depth stop further comprising a threaded fastener engaged within the threaded securement aperture such that the threaded fastener is capable of being moved into contact with the cannula shaft in order to secure the adjustable depth stop relative to the cannula shaft.

19. The PCNL access assembly of claim 16, wherein the adjustable slider includes a first leaf having a first aperture sized to accommodate the cannula shaft and a second leaf having a second aperture sized to accommodate the cannula shaft, the first leaf and the second leaf joined via a living hinge;
the first leaf is biased to a position relative to the second leaf such that the first aperture is at least partially misaligned with the second aperture in order to secure the adjustable depth stop relative to the cannula shaft; and
the first leaf and the second leaf are capable of being squeezed together to move the first aperture into alignment with the second aperture such that the adjustable depth stop may be moved relative to the cannula shaft.

20. A percutaneous nephrolithotomy (PCNL) needle, comprising:
a cannula including a shaft defining a cannula lumen extending through the shaft and a cannula hub coupled to a proximal end of the cannula;
a depth guide disposed on an outer surface of the cannula shaft, the depth guide configured to provide an indication of insertion depth;
a stylet disposable within the cannula lumen, the stylet including a tapered point at a distal end of the stylet and a stylet hub coupled to a proximal end of the stylet, the stylet hub configured to be threadedly engageable with the cannula hub;
a first alignment marker disposed on the cannula hub and a second alignment marker disposed on the stylet hub, the first alignment marker and the second alignment marker positioned to provide a visual indication when the stylet hub is aligned with the cannula hub; and
an adjustable depth stop releasably securable to the cannula shaft at a desired position relative to the depth guide, the adjustable depth guide capable of being manipulated between an adjustment configuration in which the adjustable depth guide is moveable relative to the cannula shaft and a secured configuration in which the adjustable depth guide is secured relative to the cannula shaft.
